(19) United States
(12) Patent Application Publication
(10) Pub. No.: US 2014/0295461 A1
(43) Pub. Date: Oct. 2, 2014

(54) Method for Determining Lung Injury Using Desmosine and Isodesmosine as Biomarkers

(71) Applicants: Jerome Owen Cantor, Brooklyn, NY (US); Bronislava Shteyngart, Brooklyn, NY (US)

(72) Inventors: Jerome Owen Cantor, Brooklyn, NY (US); Bronislava Shteyngart, Brooklyn, NY (US)

(21) Appl. No.: 14/222,644

(22) Filed: Mar. 23, 2014

Related U.S. Application Data
(60) Provisional application No. 61/805,564, filed on Mar. 27, 2013.

(51) Int. Cl. G01N 33/68

(52) U.S. Cl. CPC G01N 33/6884
USPC 435/7.92; 436/501; 250/282

(57) ABSTRACT

The subject invention is directed to a method for biochemically determining the extent of emphysematous changes in the lung. This method is entirely novel in that it links a biochemical parameter (the ratio of free to peptide-bound desmosine and isodesmosine [DID]) to specific morphological changes in lung tissue consisting of airspace enlargement and rupture of alveolar walls. While the total amount of DID in blood, urine, and sputum have been previously used to measure the rate of breakdown of lung elastic fibers, recent insights using percolation theory, have shown that the ratio of free to peptide-bound DID specifically reflects the total amount of elastic fiber damage in the lung, and is therefore a sensitive measure of emphysematous changes in the lung (which are the direct result of elastic fiber breakdown in alveolar septa). Furthermore, this new method establishes a relatively narrow range of values for normal individuals (without lung disease), which has not been previously possible by measuring total levels of DID (due to their large variability). The technique may be used to determine the amount of pulmonary emphysema in patients with chronic obstructive lung disease, and may also serve as a screening test for healthy smokers and other asymptomatic individuals (e.g. those with alpha-1 antitrypsin deficiency) who are at risk for developing pulmonary emphysema. Early detection of lung elastic fiber injury will result in more timely therapeutic intervention to slow the progression of the disease.

### RELATED APPLICATIONS

This application claims benefit of provisional patent No. 61/805,564, filed on Mar. 27, 2013.

### BACKGROUND OF THE INVENTION

Percolation theory is a powerful tool for modeling diverse phenomena, from electrical conductivity to the spread of infectious disease (1-3). The basic idea behind the theory is the emergence of complex macroscopic processes from the interaction of basic forces at the microscopic or even subatomic level. The theory is derived from studying the random action of fluid-like materials as they migrate through a latticework of channels. The distribution and characteristics of these channels determine the probability that they will become linked together in a network that permits the fluid to percolate from one end of the lattice to the other. This process of connecting isolated, saturated channels into a confluent stream produces an enormous variety of configurations that may interact at higher levels to form even more complex arrangements, similar to those associated with fractal geometry.

In this way, random movement through a simple matrix can generate increasing levels of complexity and establish relationships between higher and lower order phenomena. The self-similar nature of these percolation systems at different levels of scale suggests that the study of microscopic phenomena may provide insight into the macroscopic world. For this reason, percolation theory has become an attractive tool for investigating how microscopic changes in various materials affect their physical characteristics. The theory has been used to determine whether simple patterns of change in living organisms are indicative of more systemic transformations.

With regard to the lung, percolation models have been applied to the study of pulmonary diseases such as emphysema and interstitial fibrosis (4-5). Initial work used simple models to characterize the effect of mechanical forces on lung parenchyma. A network of springs (representing elastic fibers and collagen) was modified by either increasing their stiffness or removing them entirely, resulting in architectural changes similar to those seen in the lungs of patients with either pulmonary fibrosis or emphysema, respectively. Furthermore, it was noted that changes in the spring-lattice did not produce significant deformations until a critical threshold was reached, suggesting that it may be possible to predict the emergence of lung disease by examining early changes in microscopic and molecular phenomena. We will use this concept to test the hypothesis that patterns of lung elastic fiber breakdown may be a reflection of morphological changes in chronic obstructive pulmonary disease (COPD).

COPD is characterized by a low-grade, chronic inflammatory process that causes progressive deterioration of the lungs over a period of many years (6-9). The disease manifests itself clinically as persistent cough and sputum (chronic bronchitis), and by shortness of breath. The inflammatory process may extend from the airways into the lung parenchyma, resulting in the release of enzymes (elastases) that degrade the elastic fiber network surrounding alveoli. Damage to these fibers results in dilatation and rupture of the alveolar walls (emphysema) and decreases lung elastic recoil, resulting in further airflow obstruction. In the current study, we propose that patterns of elastic fiber breakdown products in body fluids may not only provide information about the loss of these fibers in the lung, but may also indicate the extent of parenchymal injury in COPD.

The elastic fiber network of the lung is composed of highly distensible, coiled proteins that provide the force necessary to expel air from the lungs during expiration (10, 11). Energy is stored within the fibers when they are distended by inhalation and is released as they recoil during exhalation. The fibers have a highly specialized structure, consisting of a core elastin protein surrounded by layers of microfibrils. The elastin molecule is composed of networks of crosslinked peptide chains containing hydrophobic domains that contribute to the elasticity of the fibers. Loss of elastic fibers, due to enzymatic or oxidative breakdown, results in uneven transmission of mechanical forces in the lung, leading to alveolar distension and rupture.

Two of the elastin crosslinks, desmosine and isodesmosine (DID), are unique to this protein and may therefore serve as a biomarker for the breakdown of elastic fibers (FIG. 1). A number of studies have shown that increased levels of these crosslinks in sputum, blood, or urine are indicative of COPD, and emphysema in particular (12-17). However, the absolute amount of desmosine and isodesmosine in body fluids may vary considerably in patients with similar levels of COPD, suggesting that the crosslinks themselves are not a useful marker for the actual extent of lung damage. For example, in one published report, DID values in the plasma of alpha-1 antiprotease-deficient patients varied from 5 to 76 ng/g protein, and did not correlate with the extent of disease, as measured by the forced expiratory volume in one second (FEV₁), expressed as a percentage of the predicted value.

Despite this finding, there is considerable evidence to support the role of crosslinks in determining the mechanical and morphological characteristics of lung parenchyma. In one study, hamsters treated with a crosslink inhibitor (beta-aminopionitrile) to modify cadmium chloride-induced lung injury had emphysematous changes in their lungs, whereas untreated animals developed interstitial fibrosis (18).

### Percolation Theory and Lung Elastic Fiber Damage

To improve the prognostic value of the DID biomarker, we will attempt to relate changes in lung mechanical forces to the release of specific elastic fiber breakdown products. Damage to these fibers will be modeled by using a mechanical network composed of interconnecting units with two different levels of stiffness (K1 and K2), corresponding to either structurally weak or strong elastic fibers, respectively (19). The amount of mechanical deflection in response to a force is inversely proportional to the degree of stiffness. Thus, K1 units are more prone to mechanical stretching than their stiffer counterparts.

The two types of units are arranged randomly throughout a 3-dimensional lattice, simulating diffuse elastic fiber damage. Under these conditions, the percolation of mechanical forces through the lattice depends on the ratio of K1 to K2 (FIG. 2). When there are few K1 units, percolation of mechanical forces is predominantly through K2, ensuring that there is little or no disruption of lung architecture. Conversely, when K1 units predominate, mechanical forces mainly percolate along the weaker pathways in the network, producing distortion of lung architecture. The ratio of K1 to K2 units is thus the critical determinant in modeling the emergence of pulmonary emphysema. When a critical ratio is reached, the disease will shift from a latent to an active state, characterized by changes in FEV₁ and other parameters.

The self-similar nature of percolation systems further suggests that the K1 to K2 ratio may reflect a larger pattern of change that extends to the microstructure of elastic fibers, where crosslinks provide tensile strength (FIG. 3). At this lower level of scale, weakly crosslinked portions of elastin peptides would be analogous to K1 fibers, whereas well-crosslinked regions would correspond to K2 fibers. In terms of breakdown products, the percolation of forces through the weakly crosslinked regions of elastin would cause greater distention of the peptide chains, increasing the likelihood of mechanical failure and breakage of chemical bonds. The unraveling of these weakly crosslinked regions would presumably make them more susceptible to enzymatic breakdown, which might result in the release of smaller peptide fragments and individual DID crosslinks (20). In contrast, the well-crosslinked regions should be less vulnerable to mechanical failure and enzymatic digestion. Consequently, their breakdown products might consist of larger peptides and less free crosslinks.

### Empirical Data:

While events at the molecular level may be difficult to confirm experimentally, there is indirect evidence that lung elastic fiber damage is accompanied by increased release of free DID crosslinks. Measurements of free and peptide-associated (bound) DID revealed a significant increase in the free/bound ratio in separate groups of COPD patients, with and without alpha-1 antiprotease deficiency, compared to normal individuals (14). Furthermore, there was a strong negative correlation between FEV₁ and the free/bound DID ratio in the combined COPD and normal population (FIG. 4). At FEV₁ values below 50% of predicted, there were several relatively low free/bound DID values, which could possibly reflect a decline in the rate of disruption of the pulmonary elastic fiber network due to a predominance of the structurally weak K1 component.

A tight clustering of free/bound DID values was seen in the group without COPD despite a large degree of variance in the absolute amounts of these breakdown products. This finding suggests that patterns of degradation are more important than individual values, which is an expected outcome if percolation processes are active. The ratio of free to bound DID may therefore be reflective of the ratio of weak to strong elastic fibers at a higher level of scale, which in turn may be indicative of the proportion of structurally weak alveolar septa at the macroscopic level. However, it should be emphasized that separate thresholds may exist at each level of scale, below which there is no apparent abnormality.

The major thesis of this discussion, that percolation theory may be a powerful tool for identifying relationships between seemingly disparate phenomena, is one that warrants further investigation, not only with regard to COPD, but other lung diseases as well. Whereas the mechanical percolation is model is suitable for lung diseases involving architectural changes in lung parenchyma, other models may be more appropriate for diseases such as pneumonia and respiratory distress.

With regard to COPD, biomarkers involving structural elements of the lung may provide more information than those associated with inflammatory processes because they reflect the dynamics of percolation processes (21, 22). In addition to the ratio of free to bound DID, relationships between elastin peptides in body fluids may also have predictive potential. For example, the proportion of lower molecular weight elastin peptides may possibly increase as the mechanical forces in the elastic fiber network become more disruptive. Using advanced assay techniques, such as mass spectrometry, it may be possible to characterize both the amount and molecular weight of individual elastin peptides and to correlate changes in their distribution with accepted parameters of COPD progression, such as FEV₁ and high-resolution computerized tomography (23).

Other pulmonary diseases involving architectural changes, such as interstitial fibrosis, may also be amenable to modeling with mechanical percolation networks. In this disorder, there is an accumulation of connective tissue in the interstitium, so one might expect to see different relationships between structural biomarkers. Whereas COPD may be associated with an increase in the ratio of free to bound DID, the opposite may be true in the case of pulmonary fibrosis, since the relative amounts of elastic fibers corresponding to K1 and K2 units would be reversed. Other connective tissue components could also provide useful biomarkers for this disease. For example, as lung expansion becomes more restricted, the ratio of more rigid (type 1) to less rigid (type 3) collagen fibers would increase, and this change might be reflected at the molecular level by the relative amounts of free and bound crosslinks derived from the turnover of collagen in the lung (24).

Percolation theory provides a “big picture” analysis of dynamic processes, relating events at different levels of scale. While the application of the theory to lung disease is at an early stage, the current study has shown that it can be an important means of identifying novel relationships between various disease phenomena. Furthermore, the use of percolation systems to model lung disease suggests that the best biomarkers are those that actually reflect the specific dynamics of the disease process. In the case of COPD, inflammatory markers may be less reliable than DID because they do not reflect the mechanical forces that are responsible for airway obstruction, and may not be involved in the self-similar pattern of changes that are an important feature of percolation systems. Consequently, their prognostic value with regard to the progression of COPD may be more limited than that of structural biomarkers such as DID. Determining which markers are most useful will play a critical role in identifying those at risk for developing this disease and will accelerate the testing of potential therapeutic agents.

### References

1. Bunde A, Kantelhart JW. Diffusion and conduction in percolation systems. In (Heitjans P, Karger J, eds) Diffusion in Condensed Matter, Springer, Berlin, 2005, pp 895-914.
2. Redner S. Fractal and multifractal scaling of electrical conduction in random resistor networks. In (Meyers, PA, ed) Encyclopedia of Complexity and Systems Science, Springer, Berlin, 2009, pp 3737-3754.
3. Sander L M, Warren C P, Sokolov I M, Simon C, Koopman J. Percolation on heterogeneous networks as a model for epidemics. Mathematical Biosciences 180 (2002) 293-305.
4. Suki B, Bates J H. Lung tissue mechanics as an emergent phenomenon. J Appl Physiol. 2011; 110(4):1111-8.
5. Bates J H, Davis G S, Majumdar A, Butnor K J, Suki B. Linking parenchymal disease progression to changes in lung mechanical function by percolation. Am J Respir Crit Care Med. 2007; 176(6):617-23.
6. Hogg J C, Timens W. The pathology of chronic obstructive pulmonary disease. Annu Rev Pathol. 2009; 4:435-59.
7. Barnes P J. New concepts in chronic obstructive pulmonary disease. Annu Rev Med. 2003; 54:113-29.
8. Tuder R M, Petrache I. Pathogenesis of chronic obstructive pulmonary disease. J Clin Invest. 2012; 122(8):2749-55.
9. Martinez F J, Donohue J F, Rennard S I. The future of chronic obstructive pulmonary disease treatment—difficulties of and barriers to drug development. Lancet. 2011; 378(9795):1027-37.
10. Li B, Daggett V. Molecular basis for the extensibility of elastin. J Muscle Res Cell Motil. 2002; 23(5-6):561-73.
11. Kielty C M, Sherratt M J, Shuttleworth C A. Elastic fibres. J Cell Sci. 2002; 115(Pt 14):2817-28.
12. Turino G M, Lin Y Y, He J, Cantor J O, Ma S. Elastin degradation: an effective biomarker in COPD. COPD. 2012; 9(4):435-8.
13. Ma S, Turino G M, Lin Y Y. Quantitation of desmosine and isodesmosine in urine, plasma, and sputum by LC-MS/MS as biomarkers for elastin degradation. J Chromatogr B Analyt Technol Biomed Life Sci. 2011 Jul. 1; 879(21):1893-8.
14. Ma S, Lin Y Y, Turino G M. Measurements of desmosine and isodesmosine by mass spectrometry in COPD. Chest. 2007; 131(5):1363-71.
15. Ma S, Lieberman S, Turino G M, Lin Y Y. The detection and quantitation of free desmosine and isodesmosine in human urine and their peptide-bound forms in sputum. Proc Natl Acad Sci U S A. 2003; 100(22):12941-3.
16. Fregonese L, Ferrari F, Fumagalli M, Luisetti M, Stolk J, ladarola P. Long-term variability of desmosine/isodesmosine as biomarker in alpha-1-antritrypsin deficiency-related COPD. COPD. 2011; 8(5):329-33.
17. Luisetti M, Ma S, ladarola P, Stone P J, Viglio S, Casado B, Lin Y Y, Snider G L, Turino G M. Desmosine as a biomarker of elastin degradation in COPD: current status and future directions. Eur Respir J. 2008; 32(5):1146-57.
18. Niewoehner D E, Hoidal J R. Lung fibrosis and emphysema: divergent responses to a common injury? Science. 1982; 217:359-60.
19. Murphy K D, Hunt G W, Almond D P. (2006): Evidence of emergent scaling in mechanical systems, Phil Mag. 2006; 86(21-22):3325-3338.
20. Jesudason R, Black L, Majumdar A, Stone P, Suki B. Differential effects of static and cyclic stretching during elastase digestion on the mechanical properties of extracellular matrices. J Appl Physiol. 2007; 103(3):803-11.
21. Doyle T J, Pinto-Plata V, Morse D, Celli B R, Rosas 10. The expanding role of biomarkers in the assessment of smoking-related parenchymal lung diseases. Chest. 2012; 142(4):1027-34.
22. Rosenberg S R, Kalhan R. Biomarkers in chronic obstructive pulmonary disease.

Transl Res. 2012; 159(4):228-37.
23. Cleverley J R, Muller N L. Advances in radiologic assessment of chronic obstructive pulmonary disease. Clin Chest Med. 2000; 21(4):653-63.
24. Last J A, Siefkin A D, Reiser K M. Type I collagen content is increased in lungs of patients with adult respiratory distress syndrome. Thorax. 1983; 38(5):364-8.

### SUMMARY OF INVENTION

The subject of the invention is a method for determining the extent of damage in the lungs of a mammal, comprising the measurement of the ratio of free to peptide-bound desmosine and isodesmosine (DID) in a bodily fluid, including (but not limited to) urine, blood, saliva, and sputum. The measurement may be performed on asymptomatic humans to screen them for the possibility of lung damage due to cigarette smoke (primary or secondary) or another injurious agent. It may also be used to determine the extent of lung damage in a person with chronic obstructive lung disease, a disease complex that includes asthma, bronchitis , or pulmonary emphysema. Other lung diseases for which this measurement may be useful include (but are not limited to) respiratory distress syndrome (both adult and neonatal forms), interstitial pulmonary fibrosis, cystic fibrosis, diffuse lung damage, pneumonia, and neoplasia.

### BRIEF DESCRIPTION OF FIGURES

FIG. 1:

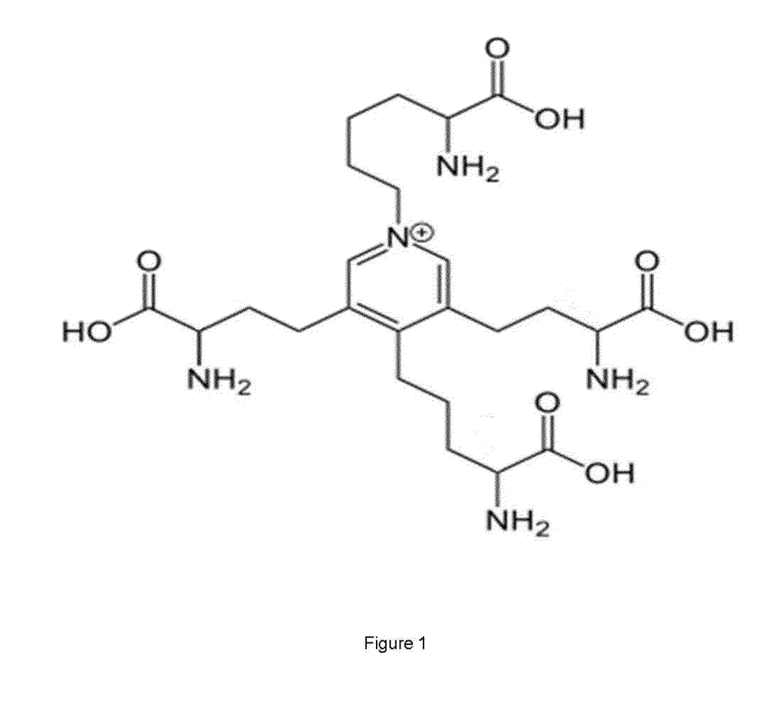

Desmosine crosslinks (shown above) are formed by the condensation of lysine side-chains on adjacent elastin peptides. Isodesmosine differs only in the location of the lysyl arms on the central pyridinium ring.

FIG. 2:

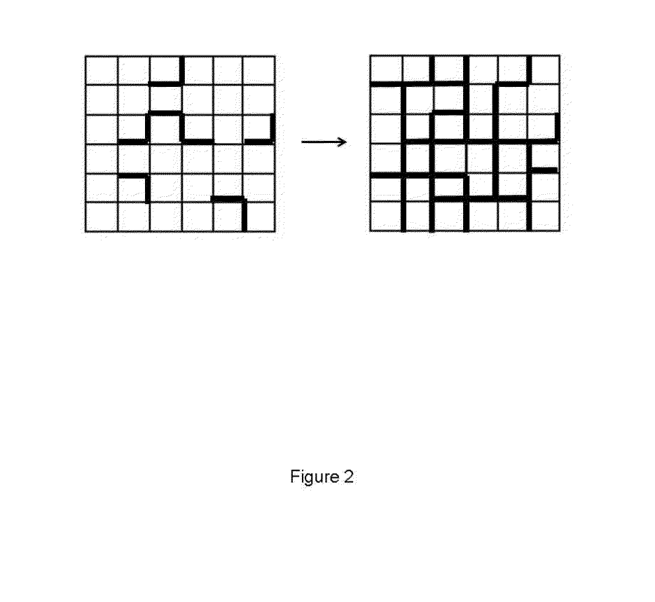

The transition from a lattice with few K1 (bold) connections to one with many of these links results in increased percolation of mechanical forces through the weaker portions of the network, thereby enhancing mechanical deflection. While this process is depicted in 2 dimensions, the actual model would involve a 3-dimensional system that more closely approximates the lung elastic fiber network.

FIG. 3:

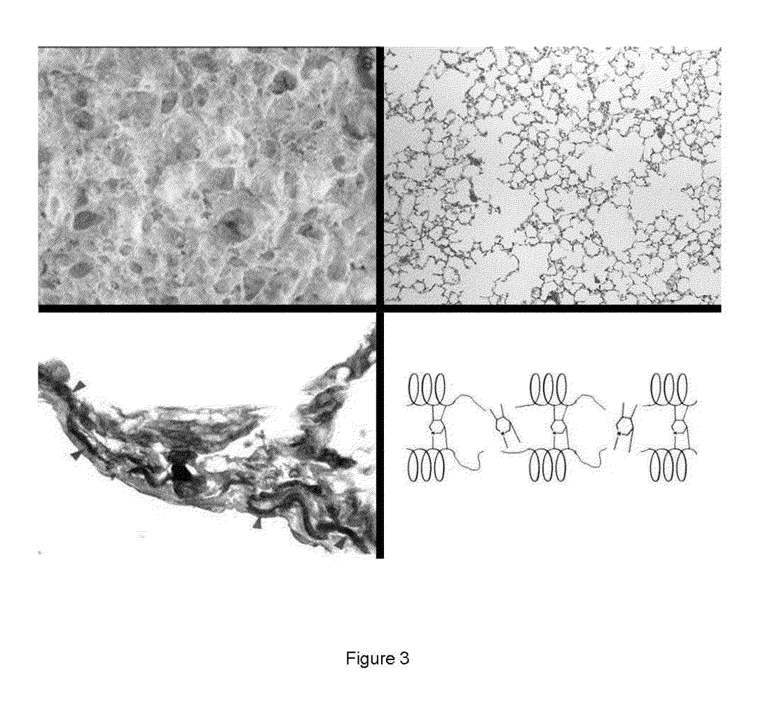

The self-similar nature of percolations systems suggests that analogous events occur at different levels of scale. The visible damage to lung parenchyma (upper left) is reflected at the low-power microscopic level by ruptured alveolar walls (upper right) and, at the high-power level, by fragmented elastic fibers (arrowheads, lower left). At the molecular level, an analogous process would be the breakage and release of elastin crosslinks (lower right).

FIG. 4:

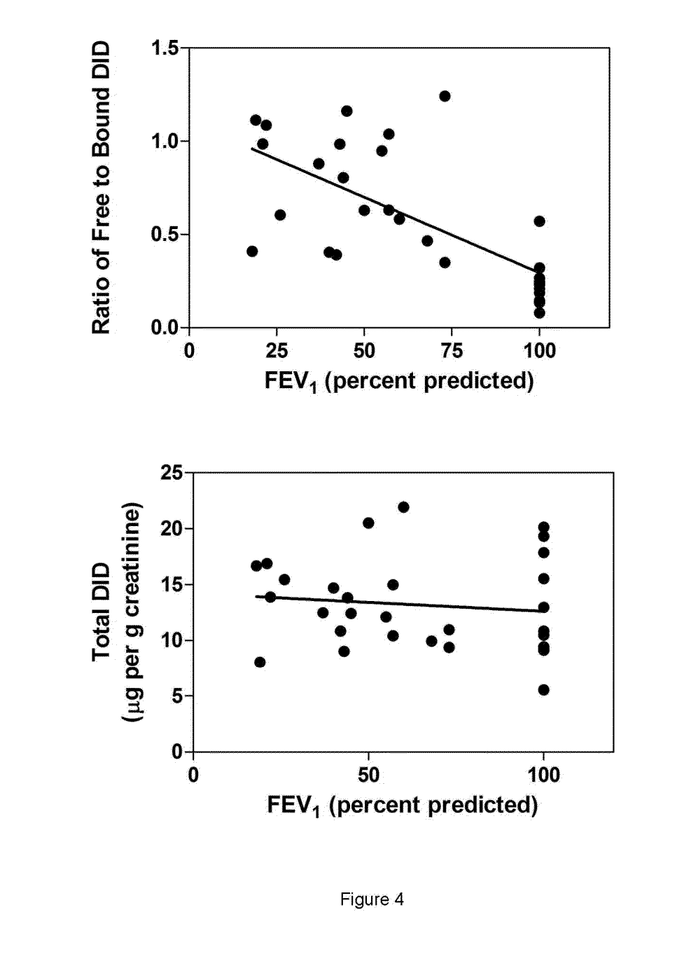

(Upper graph) There is a strong negative correlation (p<0.0001) between the ratio of free to peptide-bound urinary DID and FEV₁ in a combined population of COPD patients, with and without alpha-1 antiprotease deficiency (n=12, n=7, respectively) and normal individuals (n=11). (Lower graph) In contrast, there was no correlation between total urinary DID and FEV₁ in the same population. Due to the fact that FEV₁ data for the normal population was unavailable, they were arbitrarily assigned a value of 100 percent.

### DETAILED DESCRIPTION OF INVENTION

The subject of the invention is a method for determining the extent of damage in the lungs of a mammal, comprising the measurement of the ratio of free to peptide-bound desmosine and isodesmosine (DID) in a bodily fluid, including (but not limited to) urine, blood, saliva, and sputum. The measurement may be performed on asymptomatic humans to screen them for the possibility of lung damage due to cigarette smoke (primary or secondary), another injurious agent, or a genetic abnormality such as alpha-1 antitrypsin deficiency. It may also be used to determine the extent of lung damage in a person with chronic obstructive pulmonary disease (COPD), a disease complex that includes asthma, bronchitis , or pulmonary emphysema. Other lung diseases for which this measurement may be useful include (but are not limited to) respiratory distress syndrome (both adult and neonatal forms), interstitial pulmonary fibrosis, cystic fibrosis, diffuse lung damage, pneumonia, bronchiectasis, and neoplasia.

The measurement of the ratio of free to bound DID may be performed by a number of different techniques (either alone or in combination with one another), including chromatography, immunoassay, electrophoresis, UV spectrometry, visible light spectrometry, infrared spectrometry, fluorescence spectrometry, and mass spectrometry, including liquid chromatography-tandem mass spectrometry or dedicated mass spectrometry (which may involve the use of a hand-held dedicated mass spectrometer).

In the preferred embodiment, a sample of urine from a mammal will be divided into two equal parts. One part will be directly measured for the amount of free DID, using a combination of liquid chromatography and mass spectroscopy (LC/MS). The second part will be hydrolyzed with an acid or other reagent to release the DID associated with elastin peptides, and then subjected to the same measurement procedure to determine the total amount of DID in the sample. The amount of free DID will then be subtracted from the total DID to obtain the ratio of free to peptide-bound DID. The formula for this calculation is given below:

$${Ratio}_{( free / bound )} = \frac{( {DID}_{free} )}{( {DID}_{total} ) - ( {DID}_{free} )}$$

This ratio will then be compared to that associated with healthy mammals to determine the extent of lung disease. The normal range of values for the free to peptide-bound DID ratio will be determined by using standard statistical techniques involving the establishment of a mean value for a random population of healthy subjects and the associated standard deviation of the mean.

In another embodiment, both the free and total DID values may be normalized to one of the following parameters associated with the test sample: total protein, total creatinine, total albumin, total volume, total mass, or other appropriate parameter. The ratio of free to peptide bound DID may then be calculated irrespective of initial sample size.

In a further embodiment, the amount of free and total DID may be determined by combining different techniques. For example, free DID may be determined by mass spectrometry, while total DID may be measured by an immunoassay, such as an enzyme-linked immunosorbent (ELISA) assay. The use of an immunoassay may avoid the need for time-consuming hydrolysis of the test sample to determine total DID.

The establishment of a mean value for individuals without lung disease will pave the way for rapid, economical screening of populations at risk for development of pulmonary emphysema. To date, the only means of testing for this disease has involved the costly use of pulmonary function studies or high-resolution computerized tomography. Consequently, the subject invention represents a breakthrough in the diagnosis and management of pulmonary emphysema.

What is claimed is:

1. A method of quantifying the severity of lung disease in a mammal comprising determination of the ratio of free to peptide-bound desmosine and isodesmosine (DID) in a bodily fluid of said mammal.

2. A method of claim 1, wherein the mammal is a human.

3. A method of claim 1, wherein the mammal is an asymptomatic subject being screened for the possibility of lung disease due to one or more of the following risk factors: cigarette smoking, exposure to second-hand smoke, or a genetic disease involving lung elastic fiber injury, such as alpha-1 antitrypsin deficiency.

4. A method of claim 1, wherein the mammal may be suffering from one or more of the following lung diseases: chronic obstructive pulmonary disease (COPD), asthma, bronchitis, pulmonary emphysema, respiratory distress syndrome (adult or neonatal), interstitial pulmonary fibrosis, cystic fibrosis, diffuse alveolar damage, pneumonia, bronchiectasis, or neoplasia.

5. A method of claim 1, wherein the determination of the ratio of free to peptide-bound DID includes one or more of the following techniques: liquid chromatography, gas chromatography, immunoassay, electrophoresis, UV-spectrometry, visible light spectrometry, infrared spectrometry, fluorescence spectrometry, or mass spectrometry (including liquid chromatography-tandem mass spectrometry).

6. A method of claim 1, wherein the bodily fluid is one of the following: urine, blood, sputum, or saliva.
